(19) United States
(12) Patent Application Publication
(10) Pub. No.: US 2006/0190033 A1
(43) Pub. Date: Aug. 24, 2006

(54) Surgical clamps

(76) Inventor: Henry Pierce Molloy, Edenton, NC (US)

Correspondence Address: WILLIAMS MULLEN, FOUNTAIN PLAZA THREE, SUITE 200, 721 LAKEFRONT COMMONS, NEWPORT NEWS, VA, 23606, US

(21) Appl. No.: 11/360,790

(22) Filed: Feb. 23, 2006

Related U.S. Application Data
(60) Provisional application No. 60/655,472, filed on Feb. 23, 2005.

(51) Int. Cl. A61B 17/28

(52) U.S. Cl. 606/205

(57) ABSTRACT

A new surgical clamp which allow for approximating and everting the skin edges around a wound or incision. A surgical clamp is provided having a pair of intersecting arms and a pivot interconnecting the arms for providing rotating motion about the pivot. The arms each comprise a handle portion, a jaw portion, and a shaft portion between the handle and jaw portions. The handle portion further comprises a finger loop and a latching mechanism opposite the finger loop. The jaw portion also preferably has a skin engagement surface comprising one or more grasping teeth and a skin eversion channel. The skin engagement surfaces on each jaw portion is operably opposed and the latching mechanism operably engageable when the handle portions are rotated toward each other for holding the handle portions in a fixed position, and for operably engaging the skin engagement surfaces and the skin eversion channel.

### CROSS REFERENCE TO RELATED APPLICATION

The present application claims priority from U.S. Provisional Application Ser. No. 60/655,472, filed Feb. 23, 2005.

### BACKGROUND OF THE INVENTION

1. Field of the Invention

The present invention relates to skin approximators, namely a clamp for closing small incisions using skin adhesives.

2. Description of Related Art

Many devices and methods are known for closing skin incisions. Their purpose is to draw the skin edges together from opposite sides of a wound, to permit the wound to be closed by bandaging, suturing, or stapling. A problem with the prior art devices is that many are only capable of butting the skin edges together. It is known, however, that this does not produce as neat a final scar as is obtained when the skin edges are everted, i.e. bent outwardly (relative to the skin surface) so that they in effect form a slight “bead.” Eversion leaves the person closing the wound with the choice of simply butting the skin edges with the aid of the approximator, leaving the patient to accept the less attractive scar tissue development which results from this, or to attempt to evert the skin edges by hand which is difficult if not impossible to accomplish.

U.S. Pat. No. 6,176,868 uses elongate elements joined by a spring and placed on either side of the incision. The spring biases the elongate elements towards the incision, thus forcing the skin edges towards each other resulting in an everted closure.

U.S. Pat. No. 4,506,669 is a two-handled device with barbs extending from each handle. The barbs are forced into the skin on opposite sides of the incision. A hinge located between the barbed elements forces the barbs to rotate slightly as the handles are forced together, thus turning the skin edges upward slightly such that an everted closure is achieved.

Of the incision closure devices and methods invented, there are none that allow access to the incision while the skin edges are in the proper position, making it difficult to secure the edges in the proper configuration.

The present invention provides a device which everts the edges of an incision to allow closure of the incision using, for example, skin adhesive.

An object of the present invention is to provide a surgical clamp for closing incisions which everts the skin edges as the incision is closed.

An object of the present invention is to provide a surgical clamp for closing incisions which allow access to the closed incision such that the everted edges can be secured.

An object of the present invention is to provide a surgical clamp for closing incisions which can be manufactured as a single use device or as a reusable device.

Finally, it is an object of the present invention to accomplish the foregoing objectives in a simple and cost effective manner.

### SUMMARY OF THE INVENTION

The present invention addresses these needs by providing a surgical clamp for approximating the skin edges of a wound or incision. The claim consists of a pair of intersecting arms which connect at a pivot point and which rotate about a the pivot point. The arms include a handle portion, a jaw portion and a shaft portion between the handle and jaw portions. The jaw portion includes an engagement element which frictionally engages skin and an eversion structure for everting the skin edges. In the preferred embodiment, the engagement element is horizontal or vertical teeth which noninvasively grasp the skin. The preferred eversion structure is a channel formed along each jaw portion. The jaw portions may be moved into close proximity by operation of the handle portions in order to approximate and evert the skin edges between the jaw portions. The preferred handle is a finger loop provided on each intersecting arm. If desired, the surgical clamp may include a latch for locking the intersecting arms in a desired orientation, normally that orientation which retains the jaw portions in close proximity. The jaw portion may be perpendicular to, parallel with or at a slight angle to the shaft portion. In use, the jaw portions are placed in an open configuration by moving the handle portions away from each other. Each jaw portion is then engaged with the skin on opposite sides of the wound or incision. The handle portions are then moved towards each other, thus moving the jaw portions towards each other, such that the skin edges are approximated by the engagement element and the skin edges are everted by the eversion structure. If a latch is provided, the handle portions may then be latched, thus maintaining the skin edges in the approximated and everted configuration. If desired, a skin adhesive is then applied to the skin edges while the skin edges are held in place by said surgical clamp.

### BRIEF DESCRIPTION OF THE DRAWINGS

A more complete description of the subject matter of the present invention and the advantages thereof, can be achieved by the reference to the following detailed description by which reference is made to the accompanying drawings in which:

FIG. 1 is a perspective view of the preferred embodiment of a surgical clamp for closing incisions according to the present invention;

FIGS. 2 is a front perspective view of one element of the two that comprise the preferred embodiment of a surgical clamp for closing incisions according to the present invention;

FIG. 3 is a side perspective view of one element of the two that comprise the preferred embodiment of a surgical clamp for closing incisions according to the present invention;

FIG. 4 is a top view of an alternate embodiment of a surgical clamp for closing incisions according to the present invention;

FIG. 5 is a side view of the tip portion of an alternate embodiment of a surgical clamp for closing incisions according to the present invention;

FIG. 6 is a bottom view of an alternate embodiment of a surgical clamp for closing incisions according to the present invention;

FIGS. 7a and 7b are a cross-sectional views of the tip portion according to the present invention taken across line VII-VII in FIG. 2 or 4; and

FIG. 8 is a view of an alternate embodiment of the present invention.

### ELEMENT LIST

10 surgical clamp

12 arm elements

14 pivot point

16 finger loop

18 jaw

20 teeth

22 indentation

24 latching mechanism

### DETAILED DESCRIPTION OF THE PREFERRED EMBODIMENT

The following detailed description is of the best presently contemplated mode of carrying out the invention. This description is not to be taken in a limiting sense, but is made merely for the purpose of illustrating general principles of embodiments of the invention.

The present invention provides a surgical clamp for closing incisions in a noninvasive fashion while forcing eversion of the incision edges. The clamp allows access to the everted edges such that the edges can be secured using, for example, skin adhesive. This handheld clamp is designed to approximate skin edges of incisions or cuts to allow smooth skin closure when using skin adhesives instead of “interrupted” stitches or “running” stitches for skin closure. The clamp operator uses the clamp to hold skin edges in the proper position while the adhesive dries.

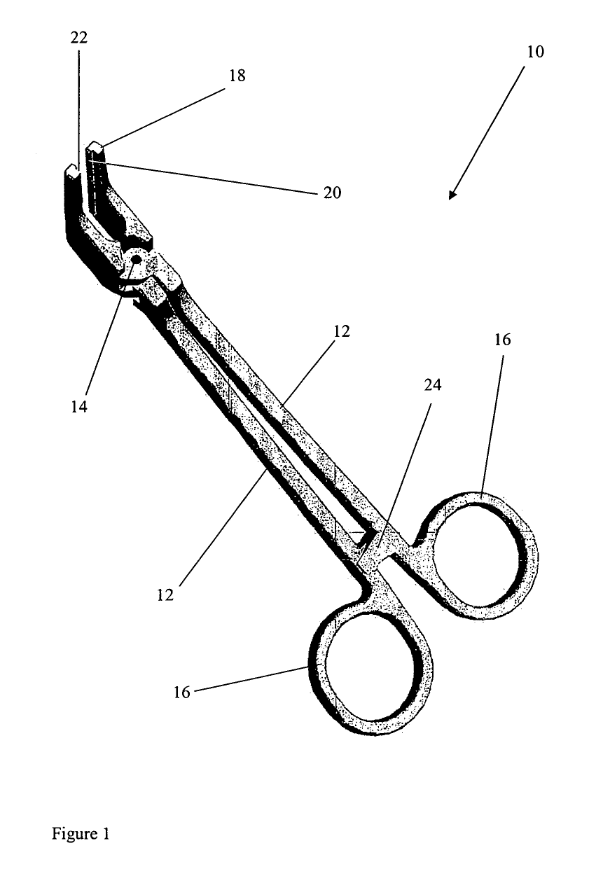

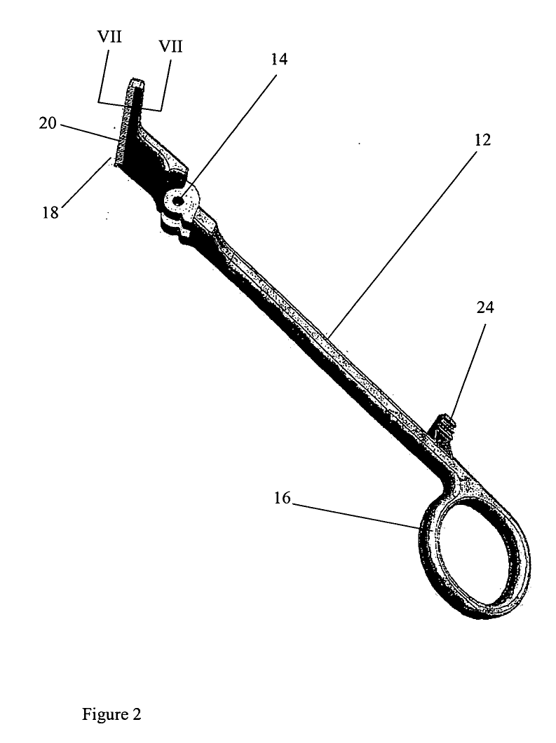

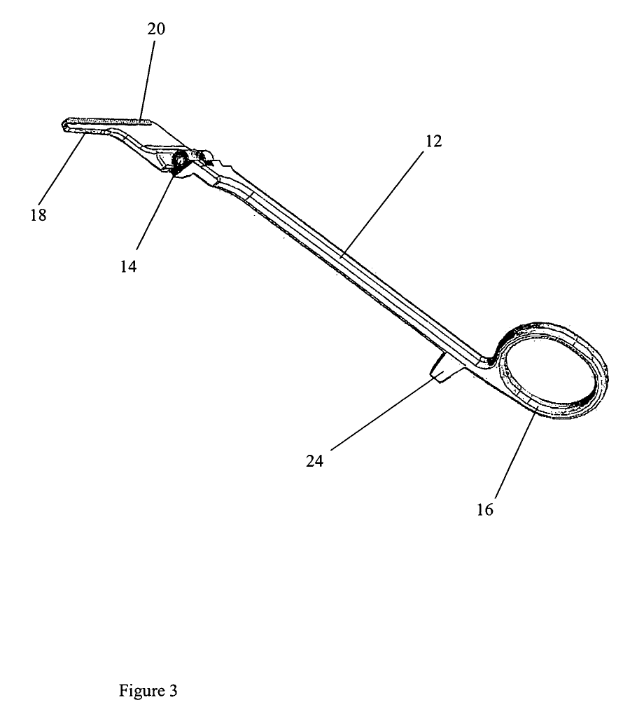

The preferred embodiment of the surgical clamp for closing incisions of the present invention is illustrated generally at 10 in FIGS. 1-3. The clamps 10 include arm elements 12 which are hinged or pivoted together at or near the lower end of the arm elements 12 at a pivot point 14 about which they can turn or pivot, in order for their opposite (lower) ends to move towards and away from one another. The pivot point 14 could, of course, be positioned in a location other than that illustrated. A “pin” or “scissor joint” connects the two pieces of the clamp 10 at the pivot point 14, allowing them to articulate like scissors.

The arm elements 12 are each provided with a handle element for controlling the arm elements 12. In the preferred embodiments, finger loops 16 are provided into which fingers of a user may be placed. The shaft portion of the arm elements 12, i.e., the portion of the arm elements 12 above the pivot point 14, are preferably approximately four inches long. The length of the entire clamp 10 allows the person holding the skin edges to keep his hand out of the field of vision and workspace of the person applying the adhesive.

In the preferred embodiment, the final approximately one inch of the arm elements 12 of the clamp 10 are bent approximately ninety degrees to form jaws 18 which are substantially perpendicular to the upper portion of the arm elements 12, allowing the operator a good working angle and leverage against the skin. The shape and angle of the clamp 10 from the pivot point 14 to the tips of the jaws 18 will allow the jaws 18 to close in a parallel configuration and the internal faces of the jaws 18 to meet. This parallel closing action does not occur with regular pivot point or box joint closing instruments.

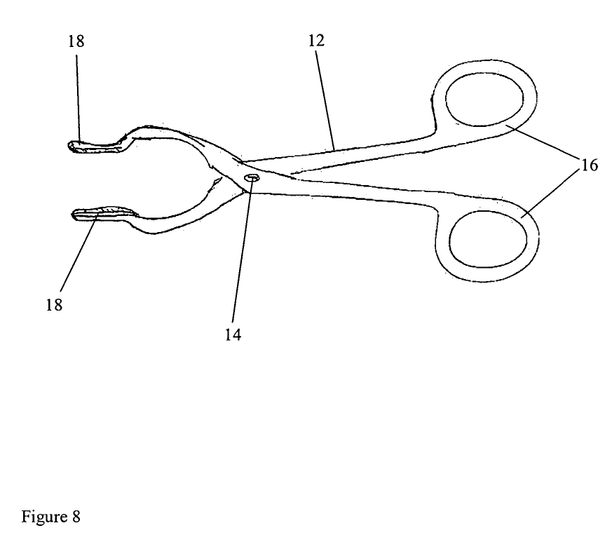

The design of the jaws 18 allows for approximation and eversion of the skin edges prior to and during application of skin adhesive to surgical wounds. To accomplish this, the internal faces of the jaws 18 have two structural characteristics, shown in detail in FIGS. 7a and 7b. The lower portion (the portion furthest away from the finger loops 16) of the internal face of each jaw 18 includes teeth 20. The teeth 20 can be formed in either the horizontal or vertical direction along the internal face of each jaw 18. The upper portion of the internal face of each jaw 18 is recessed approximately 1/32^{nd} inch along the length of the jaw 18 forming a recessed area 22 along the length of the internal face of each jaw 18. The teeth 18 help grab the skin on either side of the incision while the recessed area 22 allows the skin edges to evert for proper closure.

If desired, a latching mechanism 24 may be used to retain the arm elements 12 in a closed orientation. The preferred latching means 24 is shown in the latched position in FIG. 1, thus placing the jaws 18 in close proximity to each other. The latching means 24 of each arm element 12 is attached on the interior surface of the arm element 12. The latching means 24 of each arm element 12 is directly opposed when the two arm elements 12 of the clamp are pulled together at the finger loops 16 such that the mating surface of each latching means 24 causes the two arm elements 12 to interconnect with each other thereby engaging the teeth on each mating surface and locking the clamp in the closed position. The teeth are released by using the finger loops 16 to distort the arm elements 12 and temporarily disengaging the teeth and allowing the clamp 10 to be opened. Any latching means which can retain the arm elements in the closed orientation could be used.

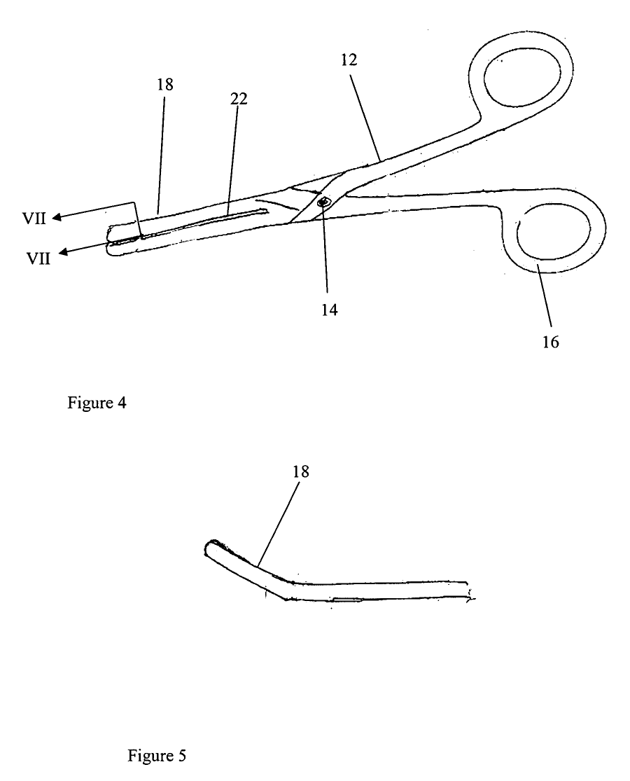

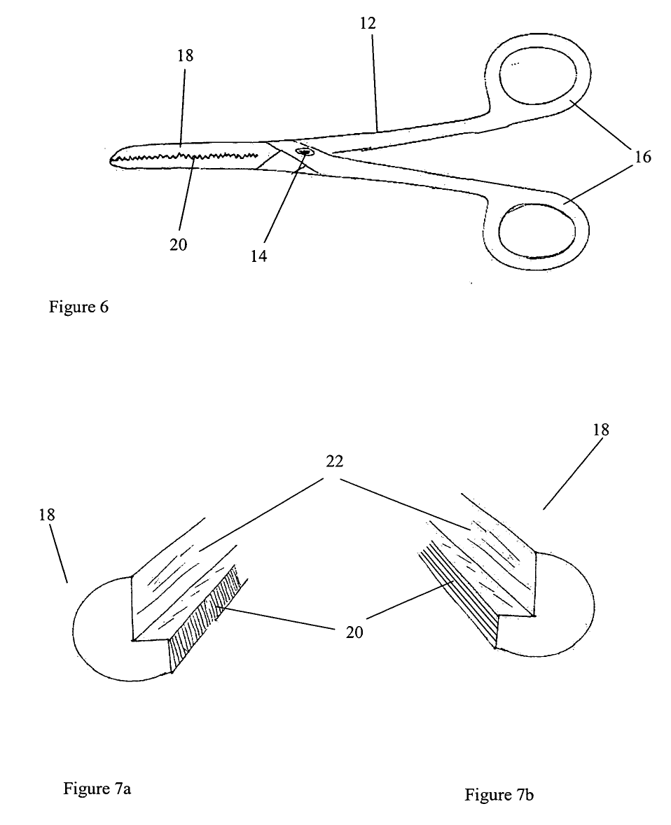

In an alternate embodiment shown in FIGS. 4-6, the final one inch or so of the arm elements 12 of the clamp 10 are bent at approximately twenty to twenty-three degrees to form jaws 18, again allowing the operator a good working angle and leverage against the skin. As described above, the internal face of each jaw 18 includes a lower portion having teeth 20 and a recessed area 22 at the upper portion of the internal face of each jaw 18. In this alternate embodiment, the jaws 18 are substantially parallel in the closed configuration although they are not in the open configuration.

In another alternate embodiment shown in FIG. 8, the arm elements 12 are curved away from each other below the pivot point 14. This allows the jaws 18 to be formed below the curve such that they are substantially parallel to each other. Again, the internal face of each jaw 18 includes a lower portion having teeth 20 and a recessed area 22 at the upper portion of the internal face of each jaw 18.

The surgical clamp according to the present invention is preferably manufactured as a one-time use disposable item. To minimize cost, the single use surgical clamp is manufactured from “pig iron” dipped in stainless steel or plastic coating. Materials for making a reusable surgical clamp include, for example, stainless steel, tempered steel, polymer plastics, and the like. Any such material sufficient for surgical use may be used to manufacture the present invention. The metal core may be tempered, or may be encased in plastic by, for example, injection molding or insert molding. The polymer used to mold the clamp can be any available extrudable polymers, such as, for example, polypropylene, polybutene, polyamides, polyamines, polymethacrylates and polymethylmethacrylates, and the like. A desirable characteristic for any polymeric material employed is resistance to high temperature deformation, which would provide for heat sterilization in the event a reusable device is desired. Those skilled in the art of manufacturing surgical clamps will be readily able to manufacture the present invention by a variety of manufacturing techniques. One arm of the clamp is attached at the end of the shaft distal to the handle portion with another similarly formed arm to form a pair of intersecting arms. The attachment of the arms is provided by a pivot means interconnecting the arms for providing a swinging motion about a common axis.

Many improvements, modifications, and additions will be apparent to the skilled artisan without departing from the spirit and scope of the present invention as described herein and defined in the following claims.

What is claimed is:

1. A surgical clamp for approximating the skin edges of a wound or incision, comprising:
a pair of intersecting arms and a pivot means interconnecting said arms for providing rotating motion about a common axis;
each of said arms comprising a handle portion, a jaw portion and a shaft portion between said handle and jaw portions;
said jaw portion further comprising an engagement means for engaging the skin and an eversion means for everting the skin edges; and
said jaw portions being movable into close proximity by operation of said handle portions such that the skin edges are approximated and everted between said jaw portions.

2. The surgical clamp as set forth in claim 1 wherein said engagement means comprises one or more grasping teeth formed on each of said jaw portions which teeth provide a frictional grasp on the skin.

3. The surgical clamp as set forth in claim 1 wherein said eversion means comprises a channel formed on each of said jaw portions which channels force eversion of the skin edges.

4. The surgical clamp as set forth in claim 1 wherein each said handle portion comprises a finger loop.

5. The surgical clamp as set forth in claim 1 further comprising a latching means for locking said intersecting arms in a desired orientation.

6. The surgical clamp as set forth in claim 1 wherein each of said jaw portions extends in a direction substantially perpendicular to said shaft portion.

7. The surgical clamp as set forth in claim 1 wherein each of said jaw portions extends in an angle between about 20 and 25 degrees from the longitudinal axis of said shaft portion.

8. A method for approximating and everting the skin edges of a wound or incision, comprising:
providing a surgical clamp which comprises a pair of intersecting arms and a pivot means interconnecting said arms for providing rotating motion about a common axis; wherein each of said arms comprises a handle portion, a jaw portion and a shaft portion between said handle and jaw portions; said jaw portion further comprising an engagement means for engaging the skin and an eversion means for everting the skin edges; and said jaw portions being movable into close proximity by operation of said handle portions such that the skin edges are approximated and everted between said jaw portions;
placing said surgical clamp in an open configuration by moving said handle portions away from each other;
placing each of said engagement means in contact with the skin surrounding the wound or incision; and
moving said handle portions towards each other such that the skin edges are approximated by said engagement means and the skin edges are everted by said eversion means.

9. The method as set forth in claim 8 further comprising latching said handle portions once they are moved towards each other and are in close proximity to each other such that the skin edges are held in an approximated and everted configuration.

10. The method as set forth in claim 8 wherein said engagement means comprises one or more grasping teeth formed on each of said jaw portions which teeth provide a frictional grasp on the skin.

11. The method as set forth in claim 8 wherein said eversion means comprises a channel formed on each of said jaw portions which channels force eversion of the skin edges.

12. The method as set forth in claim 8 wherein each said handle portion comprises a finger loop.

13. The method as set forth in claim 8 wherein each of said jaw portions extends in a direction substantially perpendicular to said shaft portion.

14. The method as set forth in claim 8 wherein each of said jaw portions extends in an angle between about 20 and 25 degrees from the longitudinal axis of said shaft portion.

15. The method as set forth in claim 8 wherein following moving said handle portions towards each other such that the skin edges are approximated by said engagement means and the skin edges are everted by said eversion means, a skin adhesive is applied to the skin edges while the skin edges are held in place by said surgical clamp.
